(19) United States
(12) Patent Application Publication
(10) Pub. No.: US 2021/0315607 A1
(43) Pub. Date: Oct. 14, 2021

(54) DETACHABLY MOUNTABLE CAMERA ASSEMBLY FOR A CANNULA ASSEMBLY

(71) Applicant: Covidien LP, Mansfield, MA (US)

(72) Inventors: Asa G. DeBlois, New Haven, CT (US); Peter T. Collings, Shelton, CT (US); Japhet Colon-Rosario, New Haven, CT (US); Smiti Narayanan, Cupertino, CA (US); Michael S. Gallie, Stamford, CT (US); Stephanie Logan, Branford, CT (US); Adia J. Solomon, Shoreview, MN (US); Matthew T. Coralli, South Windsor, CT (US)

(21) Appl. No.: 16/844,040

(22) Filed: Apr. 9, 2020

(51) Int. Cl. A61B 17/34; A61B 17/00; A61B 1/04; A61B 1/00

(52) U.S. Cl. CPC A61B 17/3423; A61B 17/3478; A61B 17/00234; A61B 2017/00283; A61B 1/00101; A61B 2017/3486; A61B 1/042

(57) ABSTRACT

A surgical kit includes a cannula assembly and a camera assembly. The cannula assembly includes a cannula housing and a cannula member extending from the cannula housing. The cannula member defines a channel therethrough. The camera assembly includes an insertion member including an elongate shaft including a supporting member extending proximally from a distal end portion of the elongate shaft, and a camera detachably supported on a proximal end portion of the supporting member of the insertion member. The camera is configured to be coupled to an outer surface of the cannula. The camera and the insertion member are dimensioned to be received through the channel of the cannula member.

### FIELD

The disclosure relates generally to surgical instruments, and more particularly, to a detachably mountable camera assembly for a cannula assembly.

### BACKGROUND

Minimally invasive surgical procedures, including endoscopic, laparoscopic and arthroscopic procedures, have been used for introducing surgical instruments inside a patient and for viewing portions of the patient's anatomy. Forming a relatively small diameter, temporary pathway to the surgical site is a key feature of most minimally invasive surgical procedures. The most common method of providing such a pathway is by inserting a cannula assembly through the skin. The cannula assembly may include an expandable balloon configured to enhance securement of the cannula assembly to an opening in tissue.

Obturators are typically designed with a tip that may be used to form an opening through the abdominal wall. An obturator is inserted into a cannula, and then the combined obturator and cannula are together placed against the skin to be penetrated. In order to penetrate the skin, the distal end of the obturator engages the skin, which may or may not have been previously cut with a scalpel. The obturator is then used to penetrate the skin and access the body cavity. By applying pressure against the proximal end of the obturator, the tip of the obturator is forced though the skin and the underlying tissue layers until the cannula and obturator enter the body cavity. The obturator is then withdrawn. The cannula remains in place within the incision for use during the minimally invasive procedure.

A camera or endoscope is inserted through the cannula for the visual inspection and magnification of the body cavity. It is desirable for the clinician to receive a visual feedback of the surgical space without interfering with surgical instruments being used by the clinician.

### SUMMARY

In accordance with the disclosure, a surgical kit includes a cannula assembly and a camera assembly. The cannula assembly includes a cannula housing and a cannula member extending from the cannula housing. The cannula member defines a channel therethrough. The camera assembly includes an insertion member including an elongate shaft having a supporting member extending proximally from a distal end portion of the elongate shaft, and a camera detachably supported on a proximal end portion of the supporting member of the insertion member. The camera is configured to be coupled to an outer surface of the cannula. The camera and the insertion member are dimensioned to be received through the channel of the cannula member.

In an aspect, the cannula member may include a magnet configured to support the camera on the outer surface of the cannula member.

In another aspect, the magnet of the cannula member may extend along a length of the cannula member.

In yet another aspect, the magnet may be flush with the outer surface of the cannula member.

In an aspect, the cannula member may include a fixation balloon proximal of the magnet.

In another aspect, the cannula housing may include a fluid port adapted to be coupled to a fluid source. The fluid port may be in communication with the fixation balloon.

In yet another aspect, the camera may be detachably supported on the supporting member such that axial displacement of the insertion member while the camera is coupled to the outer surface of the cannula releases the supporting member from the camera.

In still yet another aspect, the supporting member may be a flexible wire.

In still yet another aspect, the supporting member may be coupled to the camera by a friction fit.

In accordance with another aspect of the disclosure, a surgical kit includes a cannula assembly and a camera assembly. The cannula assembly includes a cannula housing and a cannula member extending from the cannula housing. The camera assembly includes a camera, an annular band formed of a resilient material, and an insertion rod having an elongate shaft and supporting members extending proximally from the elongate shaft. The camera is supported on the annular band. The supporting members detachably support the annular band. The insertion rod extends through the annular band. The annular band is configured to be secured on an outer surface of the cannula member.

In an aspect, the supporting members may diametrically oppose each other.

In another aspect, the supporting members may be formed of wires.

In yet another aspect, the annular band may be transitionable between a compressed state, in which, the annular band is dimensioned to be received through a channel defined through the cannula member, and an expanded state, in which, the annular band is configured to be frictionally secured about the cannula member.

In still yet another aspect, the cannula member may define a channel dimensioned to receive the annular band and the insertion rod extending through the annular band.

In still yet another aspect, the camera assembly may further include a plurality of cameras. First and second cameras of the plurality of cameras may be diametrically opposed to each other.

In an aspect, the annular band may be formed of a translucent material.

In another aspect, the insertion rod may be a trocar.

In yet another aspect, the annular band may be formed of an elastomer, gel, or foam.

In still yet another aspect, the cannula member may include a fixation balloon.

In still yet another aspect, the cannula housing may include an inflation port adapted to be coupled to an inflation source. The fixation balloon may be in fluid communication with the inflation port.

### BRIEF DESCRIPTION OF DRAWINGS

A camera assembly for use with a cannula assembly is disclosed herein with reference to the drawings, wherein:

FIG. 1 is a perspective view of a cannula assembly for use with a camera assembly in accordance with the disclosure;

FIG. 2 is a partial side view of the camera assembly, illustrating use with the cannula assembly of FIG. 1;

FIG. 3 is a partial side view of the camera assembly of FIG. 2, illustrating displacement of an insertion rod of the camera assembly to place a camera out of the cannula assembly of FIG. 1;

FIG. 4 is a partial side view of the camera assembly of FIG. 2, illustrating attachment of the camera to the cannula assembly of FIG. 1;

FIG. 5 is a partial side view of the camera assembly of FIG. 2, illustrating detachment of an insertion rod of the camera assembly from the camera coupled to the cannula assembly of FIG. 1;

FIG. 6 is a partial side view of a camera assembly in accordance with another aspect of the disclosure, illustrating use with the cannula assembly of FIG. 1;

FIG. 7 is a partial side view of the camera assembly of FIG. 6, illustrating displacement of an insertion rod of the camera assembly to place a camera of the camera assembly out of the cannula assembly of FIG. 1;

FIG. 8 is a partial side view of the camera assembly of FIG. 6, illustrating attachment of an annular band of the camera assembly to the cannula assembly of FIG. 1;

FIGS. 9 and 10 are partial side views of the camera assembly of FIG. 6, illustrating detachment of an insertion rod of the camera assembly of FIG. 6 from the annular band; and

FIG. 11 is a partial side view of the camera assembly of FIG. 6, illustrating retraction of the insertion rod out of the cannula assembly of FIG. 1.

### DETAILED DESCRIPTION

A camera assembly for use with a cannula assembly is described in detail with reference to the drawings, wherein like reference numerals designate corresponding elements in each of the several views. As used herein, the term “distal” refers to that portion of the instrument, or component thereof which is farther from the user while the term “proximal” refers to that portion of the instrument or component thereof which is closer to the user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

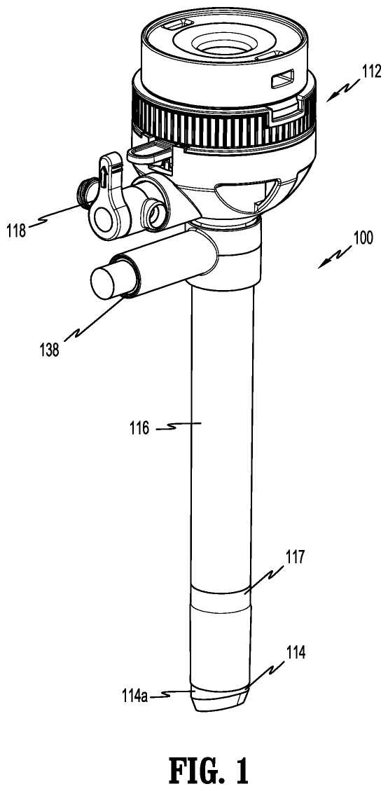

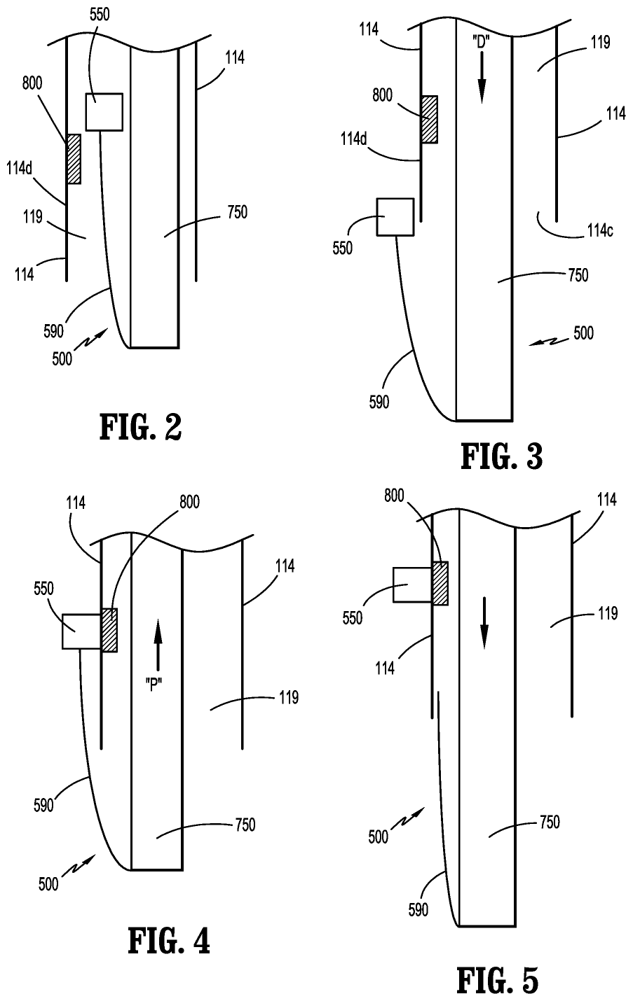

With reference to FIGS. 1 and 2, a camera assembly for use with surgical instruments, in the form of a cannula assembly 100 is shown generally as 500. The camera assembly 500 is configured to be detachably mounted on the cannula assembly 100 such that a single cannula assembly 100 may be utilized to provide visual aid to the clinician while enabling passage of surgical instruments through the cannula assembly 100. In this manner, the camera assembly 500 may reduce the number of incisions made on a patient. Furthermore, reducing the number of cannula assemblies 100 at the surgical site also maximizes the working space and reduces interference between surgical instruments.

With particular reference to FIG. 1, the cannula assembly 100 is configured to permit access to an insufflated abdominal cavity during a laparoscopic procedure to permit the introduction of a surgical object for performing various surgical tasks on internal organs within the abdominal cavity. The abdominal cavity is insufflated with fluid (e.g., CO2), to expand the body wall and provide a working space for the laparoscopic procedure. The surgical object may be a surgical instrument such as laparoscopic or endoscopic clip appliers, obturators, graspers, dissectors, retractors, staplers, laser probes, photographic devices, tubes, endoscopes and laparoscopes, electro-surgical devices and the like. The cannula assembly 100 generally includes a cannula housing 112, a cannula member 114 extending from the cannula housing 112, an outer sleeve 116 coaxially mounted over the cannula member 114, and a fixation balloon 117 mounted on the cannula member 114. The cannula assembly 100 may optionally include a locking collar (not shown) positioned about a proximal end of the cannula assembly 100 and advanceable to engage the exterior surface of tissue (e.g., the abdominal wall). The locking collar in combination with the fixation balloon 117 may minimize movement of the cannula member 114 as a surgical instrument is either inserted or withdrawn through the cannula assembly 100 and also assists in maintaining a seal about the passage in the abdominal wall.

With continued reference to FIG. 1, the cannula housing 112 is dimensioned for engagement by the clinician and may include one or more internal seals (not shown) adapted to establish a seal about a surgical object introduced therethrough. The internal seals may include, e.g., a duck bill or zero-closure seal positioned in the cannula housing 112. For example, the zero-closure seal may be formed of a suitable resilient material (e.g., silicone) and be configured to inhibit fluids from exiting proximally through the cannula housing 112 in the absence of a surgical object inserted therethrough. The cannula housing 112 and the cannula member 114 may be integrally formed as a single construct. Alternatively, the cannula housing 112 and the cannula member 114 may be monolithically formed. For example, the cannula housing 112 and the cannula member 114 may be formed of suitable biocompatible materials such as medical grade metals (e.g., stainless steel), polymeric materials (e.g., polycarbonate), or combinations thereof. The cannula member 114 may have a distal end portion 114a that is tapered.

The cannula housing 112 also may include an insufflation connector 118 (e.g., a luer connector) for connecting to a source of insufflation fluids (not shown) for delivery within, e.g., the abdominal cavity. A longitudinal lumen defined by the cannula member 114 is also in fluid communication with the insufflation connector 118 to convey insufflation fluids into the abdominal cavity to establish and/or maintain the pneumoperitoneum. The fixation balloon 117 is coupled to the outer sleeve 116 and is coaxially mounted about the distal end portion 114a of the cannula member 114. The cannula member 114 further includes a fluid port 138 in fluid communication with the fixation balloon 117. The fluid port 138 is adapted to be coupled to a source of inflation fluids to inflate the fixation balloon 117. The fixation balloon 117 expands radially outwardly upon passage of inflation fluids through the fluid port 38.

With reference now to FIG. 2, the camera assembly 500 includes a camera 550 and an insertion rod 750 detachably supporting the camera 550 by a supporting member 590 such as, e.g., a wire. The cannula member 114 includes a magnet 800 disposed on an inner surface thereof such that the camera 550 may be magnetically coupled to an outer surface 114d of the cannula member 114. Alternatively, the magnet 800 may be flush with the outer surface 114d of the cannula member 114. In particular, the magnet 800 is disposed distal of the fixation balloon 117 (FIG. 1). The camera 550 and the insertion rod 750 are dimensioned to be received through a channel 119 defined by the cannula member 114. The camera 550 may be wirelessly connected to a device (not shown) in the operating room or in a remote location to provide visual feedback of the body cavity to the clinician.

With reference to FIG. 2, the camera assembly 500 is initially placed in the channel 119 of the cannula member 114 via the cannula housing 112 (FIG. 1). With reference to FIG. 3, the insertion rod 750 is advanced (in the direction of an arrow “D”) through a distal opening 114c of the cannula member 114 such that the camera 550 is placed out of the channel 119 of the cannula member 114. Thereafter, the camera 550 is placed in registration with the magnet 800 by retracting the insertion rod 750 in the direction of an arrow “P” (FIG. 4). While the magnet 800 is shown to occupy a small portion of the cannula member 114, in order to facilitate placement of the camera 550 on the magnet 800, the magnet 800 may be, e.g., circumferentially arranged, and/or extend along a length of the cannula member 114. With reference to FIG. 5, after the camera 550 is magnetically coupled to the magnet 800, the insertion rod 750 may be advanced farther to disengage the supporting member 590 from the camera 550. For example, the supporting member 590 may frictionally support the camera 550. The magnetic coupling of the camera 550 and the magnet 800 may be stronger than the frictional coupling between the camera 550 and the supporting member 590. Specifically, once the camera 550 is magnetically coupled to the magnet 800, the insertion rod 750 may be decoupled from the cannula assembly 100. In this manner, the placement of the camera 550 on the outer surface 114d of the cannula member 114 enables the clinician to utilize the cannula assembly 100 for other surgical instruments. Further, delivery of the camera assembly 500 through the channel 119 of the cannula member 114 minimizes the size of the incision and trauma to tissue. While a single camera 550 is shown, it is contemplated that the cannula assembly 100 may utilized a plurality of cameras 550 for, e.g., a 360-degree view of the surgical space.

With reference now to FIG. 6, a camera assembly in accordance with another aspect of the disclosure for use with the cannula assembly 100 is shown generally as 700. The camera assembly 700 includes an annular band 710, a camera 720 supported in the annular band 710, and an insertion rod 745 detachably supporting the annular band 710. In particular, the annular band 710 may be formed of compressible and resilient material such as, e.g., an elastomer, gel, or foam. For example, the annular band 710 may be formed of a translucent material. The annular band 710 is dimensioned to be secured about the cannula member 114. The camera 720 is secured with the annular band 710. For example, a plurality of cameras 720 may be provided on the annular band 710 to provide a 360-degree viewing of the surgical site. The camera 720 may be wirelessly connected to a device (not shown) in the operating room or in a remote location to provide visual feedback of the body cavity to the clinician. The insertion rod 745 includes supporting members 752 diametrically coupled to the annular band 710. In particular, the supporting members 752 extend proximally from a distal end portion 755 of the insertion rod 745 such that the insertion rod 745 extends through the annular band 710. The supporting members 752 may be flexible wires. The insertion rod 745 may include a pointed tip 745a configured to cut or separate tissue. For example, the insertion rod 745 may be a trocar configured to support the annular band 710.

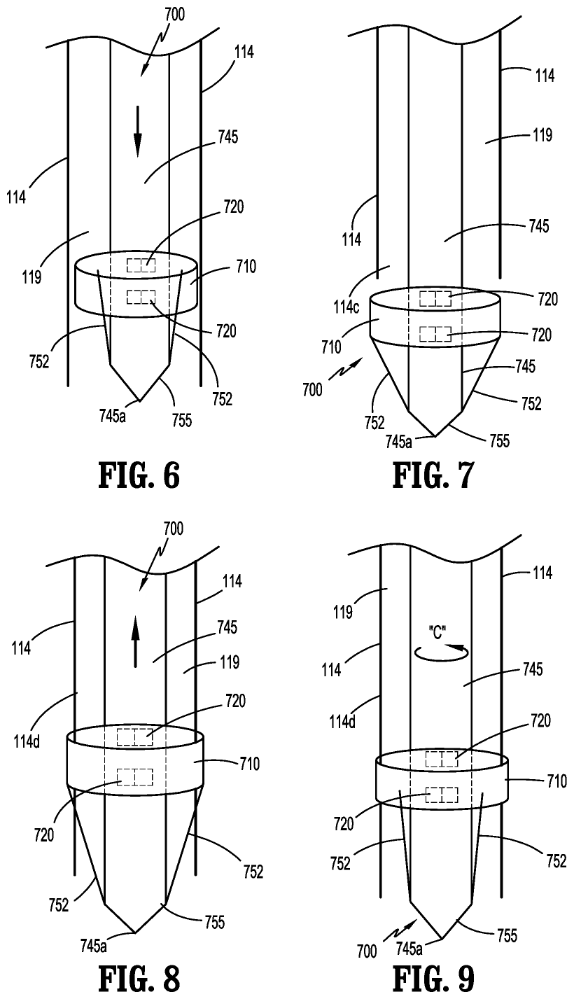

With reference now to FIGS. 7 and 8, the camera assembly 700 is placed within the channel of 119 of the cannula member 114. The annular band 710 is compressed to fit within the dimensions of the channel 119. Thereafter, the insertion rod 745 is advanced to place the annular band 710 out of the channel 119 through the distal opening 114c of the cannula member 114. Once the annular band 710 is released from the channel 119, the annular band 710 expands to its natural dimensions. Upon expansion of the annular band 710, the insertion rod 745 is retracted such that the annular band 710 is mounted about the cannula member 114. At this time, the compressible and resilient nature of the annular band secures the annular band 710 on the outer surface 114d of the cannula member 114.

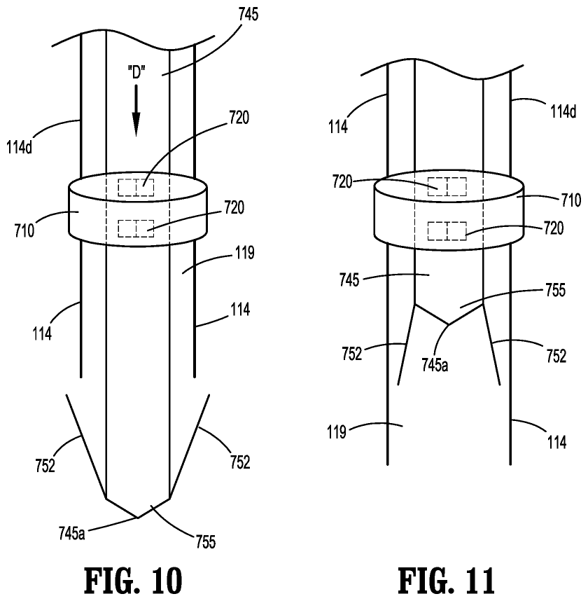

With reference now to FIGS. 9-11, the insertion rod 745 may be rotated, e.g., in the direction of an arrow “C”, and advanced in the direction of an arrow “D” to disengage the supporting members 752 from the annular band 710. The supporting members 752 may be coupled to the annular band 710 by, e.g., friction fit, ultrasonic welding, etc. to enable disengagement through rotation and advancement of the insertion rod 745. After the supporting members 752 are disengaged from the annular band 710, the insertion rod 745 may be retracted to remove the insertion rod 745 from the cannula assembly 100. During the retraction of the insertion rod 745, the supporting members 752 that are flexible are inverted to extend distally from the distal end portion 755 of the of the insertion rod 745. In this manner, the placement of the annular band 710 supporting the camera 720, on the outer surface 114d of the cannula member 114 enables the clinician to utilize the cannula assembly 100 for other surgical instruments. Further, delivery of the camera assembly 700 through the channel 119 of the cannula member 114 minimizes trauma to tissue. In addition, through the use of a compressible and resilient annular band 710, the camera assembly 700 may be used with any cannula configuration without additional support structures to accommodate the annular band 710. It is contemplated that while two diametrically opposed cameras 720 are shown, a plurality of circumferentially arranged cameras 720 may be utilized for, e.g., a 360-degree view of the surgical space.

In use, an incision is made in a body wall to gain entry to a body cavity, such as the abdominal cavity. The distal end portion 114a of the cannula member 114 of the cannula assembly 100 is inserted through the incision until at least the fixation balloon 117 is within the body cavity. The fixation balloon 117 may be inflated after the cannula member 114 is properly placed through the body wall and into the body cavity. A fluid source such as e.g., a syringe (not shown), may be connected to the inflation port 138 to supply an inflation fluid to the fixation balloon 117. The fixation balloon 117 is inflated until the fixation balloon 117 is in a sealing relation with the body wall. With the incision sealed, the body cavity may be insufflated with CO2, a similar gas, or another insufflation fluid. The camera assemblies 500, 700 may be inserted through the cannula assembly 100 and mounted on the outer surface 114d of the cannula member 114 for visual feedback of the surgical space to the clinician. Surgical instruments (not shown) may be inserted through the cannula assembly 100 to perform desired surgical procedures. To deflate the fixation balloon 117 for removal of the cannula assembly 100 from the body cavity, the inflation port 138 may be opened to release the fluid therethrough.

In this manner, utilizing camera assemblies 500, 700 to detachably mount cameras 550, 720 on the outer surface 114d of the cannula member 114, while enabling use of the channel 119 of the cannula member 114 for other surgical instruments, reduces number of cannula assemblies 100 on the patient, thereby reducing the number of incisions needed on the patient and maximizing working space for surgical instruments.

It is further contemplated that the cannula assembly 100 may be used with an obturator (not shown). The obturator generally includes a head portion having latches configured to engage respective notches defined in the cannula housing 112 of the cannula assembly 100 to enhance securement therewith, an elongate shaft extending from the head portion, and an optical penetrating tip coupled to a distal end of the elongate shaft. The optical penetrating tip may be used to penetrate the skin and access the body cavity. By applying pressure against the proximal end of the obturator, the tip of the obturator is forced though the skin and the underlying tissue layers until the cannula and obturator enter the body cavity.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A surgical kit comprising:
a cannula assembly including:
a cannula housing; and
a cannula member extending from the cannula housing, the cannula member defining a channel therethrough; and
a camera assembly including:
an insertion member including an elongate shaft having a supporting member extending proximally from a distal end portion of the elongate shaft; and
a camera detachably supported on a proximal end portion of the supporting member of the insertion member, the camera configured to be coupled to an outer surface of the cannula, wherein the camera and the insertion member are dimensioned to be received through the channel of the cannula member.

2. The surgical kit according to claim 1, wherein the cannula member includes a magnet configured to support the camera on the outer surface of the cannula member.

3. The surgical kit according to claim 2, wherein the magnet of the cannula member extends along a length of the cannula member.

4. The surgical kit according to claim 2, wherein the magnet is flush with the outer surface of the cannula member.

5. The surgical kit according to claim 2, wherein the cannula member includes a fixation balloon proximal of the magnet.

6. The surgical kit according to claim 3, wherein the cannula housing includes a fluid port adapted to be coupled to a fluid source, the fluid port in communication with the fixation balloon.

7. The surgical kit according to claim 1, wherein the camera is detachably supported on the supporting member such that axial displacement of the insertion member while the camera is coupled to the outer surface of the cannula releases the supporting member from the camera.

8. The surgical kit according to claim 1, wherein the supporting member is a flexible wire.

9. The surgical kit according to claim 1, wherein the supporting member is coupled to the camera by a friction fit.

10. A surgical kit comprising:
a cannula assembly including:
a cannula housing; and
a cannula member extending from the cannula housing; and
a camera assembly including:
a camera;
an annular band formed of a resilient material, the camera supported on the annular band; and
an insertion rod having an elongate shaft and supporting members extending proximally from the elongate shaft, the supporting members detachably supporting the annular band, the insertion rod extending through the annular band,
wherein the annular band is configured to be secured on an outer surface of the cannula member.

11. The surgical kit according to claim 10, wherein the supporting members diametrically oppose each other.

12. The surgical kit according to claim 10, wherein the supporting members are formed of wires.

13. The surgical kit according to claim 10, wherein the annular band is transitionable between a compressed state, in which, the annular band is dimensioned to be received through a channel defined through the cannula member, and an expanded state, in which, the annular band is configured to be frictionally secured about the cannula member.

14. The surgical kit according to claim 10, wherein the cannula member defines a channel dimensioned to receive the annular band and the insertion rod extending through the annular band.

15. The surgical kit according to claim 10, wherein the camera assembly further includes a plurality of cameras, first and second cameras of the plurality of cameras diametrically opposed to each other.

16. The surgical kit according to claim 10, wherein the annular band is formed of a translucent material.

17. The surgical kit according to claim 10, wherein the insertion rod is a trocar.

18. The surgical kit according to claim 10, wherein the annular band is formed of an elastomer, gel, or foam.

19. The surgical kit according to claim 10, wherein the cannula member includes a fixation balloon.

20. The surgical kit according to claim 19, wherein the cannula housing includes an inflation port adapted to be coupled to an inflation source, the fixation balloon being in fluid communication with the inflation port.
